(19) United States
(12) Patent Application Publication
(10) Pub. No.: US 2020/0121292 A1
(43) Pub. Date: Apr. 23, 2020

(54) GENERALIZED DMAS ALGORITHM FOR IMPROVED ULTRASOUND IMAGING

(71) Applicant: Olympus Scientific Solutions Americas Corp., Waltham, MA (US)

(72) Inventors: Chi-Hang Kwan, Quebec (CA); Jeremy Moriot, Quebec (CA); Charles Brillon, Quebec (CA)

(73) Assignee: Olympus Scientific Solutions Americas Corp., Waltham, MA (US)

(21) Appl. No.: 16/655,150

(22) Filed: Oct. 16, 2019

Related U.S. Application Data
(60) Provisional application No. 62/746,660, filed on Oct. 17, 2018.

(51) Int. Cl. A61B 8/08; A61B 8/00; G01S 7/52

(52) U.S. Cl. CPC A61B 8/5223; A61B 8/5207; G01S 7/52003; G01S 7/52036; A61B 8/4488

(57) ABSTRACT

Example embodiments of the present invention relate to a method and an apparatus for ultrasound imaging wherein transmitting elements of an ultrasonic array probe transmit ultrasound energy and receiving elements of the ultrasonic array probe receive received signals from the test object. The method includes deriving analytic signal values from the received signals, the analytic signal values being derived by applying a Hilbert transform to the received signals and performing a summation of multiple signal products derived by multiplication of a corresponding group of analytic signal values.

### CROSS REFERENCE TO RELATED APPLICATION

This Application claims the benefit of U.S. Provisional Patent Application Ser. No. 62/746,660 entitled “GENERALIZED DMAS ALGORITHM FOR IMPROVED ULTRASOUND IMAGING” filed on Oct. 17, 2018 the teachings of which application are hereby incorporated herein by reference in their entirety.

### FIELD OF THE INVENTION

The invention relates in general to ultrasound imaging, and in particular to an improved algorithm for obtaining ultrasound images.

### BACKGROUND OF THE INVENTION

In existing practice, ultrasound imaging using sectorial scans or the Total Focusing Method (TFM) is often performed using the Delay-And-Sum (DAS) algorithm. In the DAS algorithm, imaging is performed by simply summing received ultrasound echo signals with delays appropriate to the total length of the transmission and reception paths. The main advantage of this algorithm is that it is fast and simple to implement.

### SUMMARY OF THE INVENTION

Example embodiments of the present invention relate to a method and an apparatus for ultrasound imaging wherein transmitting elements of an ultrasonic array probe transmit ultrasound energy and receiving elements of the ultrasonic array probe receive received signals from the test object. The method includes deriving analytic signal values from the received signals, the analytic signal values being derived by applying a Hilbert transform to the received signals and performing a summation of multiple signal products derived by multiplication of a corresponding group of analytic signal values.

### BRIEF DESCRIPTION OF THE DRAWINGS

FIG. 1 is a block diagram of an ultrasound imaging system according to an example embodiment of the present disclosure.

FIG. 2A is a diagram illustrating representations of summations used for the GDMAS algorithm according to the present disclosure.

FIG. 2B shows a correlation matrix of the summations used for the GDMAS algorithm according to the present disclosure.

FIG. 3 is a photograph of an ultrasound array probe on a calibration block.

FIG. 4 shows images of a calibration block created with the DAS algorithm compared with images created with the GDMAS algorithm according to the present disclosure.

FIG. 5. is block diagram system according to an example embodiment of the present disclosure.

FIG. 6 is a diagram of an example embodiment of the present disclosure as embodied in computer program code.

### DETAILED DESCRIPTION OF PREFERRED EMBODIMENT

In traditional Delay-And-Sum (DAS), the resulting image quality suffers from several disadvantages:
Side lobes and grating lobes, caused by sound energy that spreads out from the transducer at angles other than the primary path, have significant amplitude.
The DAS algorithm does not perform any incoherent noise/sidelobe filtering prior to summation.
For a given wavelength and number of transducer array elements, the spatial resolution of the DAS algorithm is relatively poor.

Matrone et al (IEEE Trans. Med. Imag. 34(4) 2015 pp.940-949) have used an alternative to the DAS algorithm, known as Delay-Multiply-And-Sum (DMAS). DMAS is based on cross-correlation between each received signal, which has several advantages compared to DAS:
Due to the multiplication step, DMAS has intrinsic cross-correlation between signals. Such cross-correlation filters incoherent noise before the summation step, and then the coherent signal is further reinforced in the summation step. In contrast, the DAS algorithm does not filter incoherent noise/sidelobe, but only reinforces the coherent signal during the summation step. Consequently, DMAS has better noise and/or sidelobe suppression.
DMAS increases the number of signals involved in the imaging process, thereby improving the image resolution.
When using DMAS, the effective center frequency of the output signal is artificially doubled by the multiplication step, thereby improving the image resolution.

However, the DMAS algorithm suffers from a major disadvantage in that it requires far more computational operation than DAS. Whereas DAS computation time scales with N, where N is the number of waveform signals used to form an image, the computation time for DMAS scales approximately with N²/2.

There therefore exists a need for an algorithm which has image resolution equal to or better than DMAS, with computation time similar to DAS. Accordingly, it is a general objective of the present disclosure to provide an ultrasound imaging algorithm which has superior imaging resolution, equal to or better than the DMAS algorithm, with computation time similar to the DAS algorithm. The objective is achieved by deriving analytic signal values from the received signals using the Hilbert transform, and then performing a summation over the products of a number of analytic signal values for a selected subset of acquisition configurations. An acquisition configuration is defined by the delays of the transmitting and receiving elements, and the number of elements used in transmission and reception. The order of the multiplication may be raised to any real positive number Q.

An ultrasound acquisition configuration is composed of groups of transducer elements (called arrays) transmitting and receiving ultrasound signals. The transmitting and receiving arrays can be the same (pulse-echo configuration) or different (pitch-catch configuration). A delay is applied to each element of an array, and these delays are known as transmission laws when applied to a transmitting array, and reception laws when applied to a receiving array. The physical size of each array, known as an aperture, is proportional to the number of elements. Signals acquired by receiving elements in each acquisition configuration are called waveforms and are denoted s(t). To form an image, a set of N waveforms is combined with delays τ. Note that the imaging delay τ can be different from the transmission and reception delays. In addition, the number of imaging waveforms N can be smaller than the total number of waveforms acquired.

For example, in the Phased Array Ultrasound (PAUT) imaging method, the transmitting elements fire with delays according to the transmission law, and the receiving elements receive with zero delay. Therefore at each image line, the total number of waveforms N used to form the image is equal to the aperture of the receiving array. Imaging delays τ are applied before summation to form an image (e.g. B-Scan or S-Scan).

Alternatively, in the Total Focusing Method (TFM) imaging method, one transmission element fires without delay and all elements of the receiving array record without delay. This process is repeated for all elements in the transmitting array, thereby producing a Full Matrix Capture (FMC) matrix. Therefore at each image pixel, the total number of waveforms N used to form the image is equal to the product of the apertures of the transmitting and receiving arrays. Imaging delays τ are applied before summation to form a TFM image for different acoustic modes.

In yet another example, in the Plane Wave TFM imaging method, the elements of the transmitting array fire using an angled transmission law, the receiving elements receive using an angled reception law and the received waveforms are summed. This process is repeated for all transmission and reception angles to form a Plane Wave FMC. Therefore at each image pixel, the total number of waveforms N used to form the image is equal to the product of the number of transmission and reception angles. Imaging delays τ are applied before summation to form a Plane Wave TFM image for different acoustic modes.

The above examples of imaging methods are illustrative of the application of the imaging algorithm of the present disclosure. However the examples are not intended in any way to limit the application of the algorithm, which may be applied to any ultrasound configuration combining the mentioned parameters or other parameters (for example, signal apodization, or phase compensation).

Note that a given imaging method may include a number of acquisition configurations, wherein each acquisition configuration produces a waveform. The image may be formed from a selected subset of the waveforms, which may include all or some of the acquisition configurations.

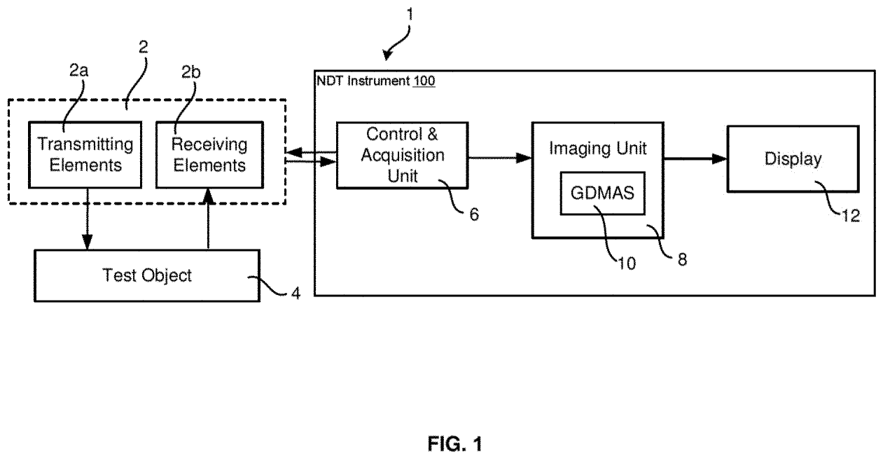

FIG. 1 is a schematic representation of an ultrasound imaging system 1 including an instrument 100. At least one array probe 2 comprises a number T of transmitting elements 2a transmitting ultrasound energy to a test object 4, and a number R of receiving elements 2b receiving echo signal responses from test object 4. Transmitting elements 2a and receiving elements 2b may be located on the same probe 2 or on different probes 2. A control & acquisition unit 6 controls ultrasound transmission from transmitting elements 2a and acquires echo signals from receiving elements 2b. Echo signals are digitized by control & acquisition unit 6 and passed to an imaging unit 8. Imaging unit 8 comprises hardware and further comprises an algorithm unit 10 configured to implement the GDMAS algorithm of the present disclosure. Imaging unit 8 passes imaging information to a display 12 which displays an image of flaws in test object 4.

Using the DAS algorithm, the algorithm output y_{DAS} is given by:

$${y}_{DAS} = {\sum }_{j = 1}^{N} {s}_{j} ( {τ}_{j} ) \qquad ( 1 )$$

where the amplitude received by a receiving element j at a delay time τⱼ is denoted by sⱼ(τⱼ).

Using the DMAS algorithm according to Matrone et al, the algorithm output y_{DMAS} is given by:

$${y}_{DMAS} = {\sum }_{j = 1}^{N - 1} {\sum }_{k = j + 1}^{N} sign ( {s}_{j} ( {τ}_{j} ) \cdot {s}_{k} ( {τ}_{k} ) ) \cdot \sqrt{\vert {s}_{j} ( {τ}_{j} ) \cdot {s}_{k} ( {τ}_{k} ) \vert} \qquad ( 2 )$$

The DMAS algorithm sums the product between the pairs of received signals indicated in the summation. The square root of the absolute value of the multiplied amplitudes is summed, but the sign of the multiplication is maintained by the sign function.

Note that, for a B-scan image, the algorithm outputs y_{DAS} or Y_{DMAS} are the individual summed A-scans, wherein the B-scan image comprises a stacking of the summed A-scans as the probe position is scanned. For a TFM image, y_{DAS} or Y_{DMAS} are the intensity values at each image pixel position.

It should be understood that, in traditional DMAS, summation occurs only for the unshaded cells where k>j. The diagonal elements where k=j (autocorrelations) are not considered in the algorithm of Matrone et al. To save computation time, the summations for jk are not repeated for kj.

It should be noted that the multiplications of the DMAS algorithm introduce a DC component and a doubled-frequency component. This can be seen in equation (3) below which is an example of multiplication of two sinusoidal waveforms of the same frequency:

A cos(ωt)·B cos(ωt)=1/2[AB+AB cos(2ωt)] (3)

It is therefore necessary for the DMAS algorithm to include a filter to remove the DC component.

The generalized DMAS (GDMAS) algorithm according to the present disclosure achieves image quality equivalent to or better than the DMAS algorithm, with computation times comparable to the DAS algorithm. The GDMAS algorithm comprises the following computational steps:
1. Deriving the analytic signals of the acquired waveforms using the Hilbert transform. The GDMAS algorithm then operates on the analytic signals rather than the real time waveforms.
2. Performing a summation over the products of each pair of analytic signals for a selected subset of the acquired waveforms. The summation may be performed for any imaging method such as B-Scan imaging or FMC TFM imaging.
3. Raising the order of the multiplication of the GDMAS method to any real positive number Q.

Note that it is step 3 which enables the algorithm to be a generalized algorithm, since the power can be raised higher than 2, and moreover the case of Q=1 corresponds to the DAS algorithm as the base case.

The steps of the GDMAS algorithm are described in more detail below.

In step 1, analytic signals of the waveforms are derived by application of the Hilbert transform:

ŝ(τⱼ)=s(τⱼ)+i [symbol] {s(τⱼ)} (4)

where H{ } is the Hilbert transform. The Hilbert transform applies a 90° phase shift to the signal. For example, [symbol] {cos(ωt)}=sin(ωt). In practice, the analytic signal is calculated by setting to zero all negative frequency components of the Fast Fourier Transform (FFT) of a signal, and then performing an Inverse Fast Fourier Transform (IFFT).

Thus, the analytic signal of a sinusoidal signal is a complex exponential:

cos(ωt)+sin(ωt)=exp(ωt) (5)

When two complex exponentials are multiplied, no DC component is present:

A exp(iωt)·B exp(ωt)=AB exp(i2ωt) (6)

Thus, unlike the DMAS algorithm, no lowpass filter is required when using the GDMAS algorithm. The need for a lowpass filter in the DMAS algorithm is a disadvantage for B-scan imaging because the frequency cutoff and order of the filter need to be adjusted to minimize distortion to other frequency components. For TFM imaging, lowpass filtering is not feasible since there are no outputted summed A-scans upon which to apply the filtering.

A further advantage of application of the Hilbert transform is that the analytic signal also allows an easy way to find the envelope of any oscillation. The absolute value of the analytic signal corresponds to the envelope of the oscillations, and such knowledge of envelopes is useful because by comparing envelopes a user may easily find the scan locations with the maximum flaw scattering amplitude.

In step 2 of the GDMAS algorithm, a summation is performed over the products of each pair of analytic signals for a selected subset of the acquired waveforms:

$$\begin{matrix}{y}_{GDMAS} = {\sum }_{j = 1}^{N} {\sum }_{k = 1}^{N} {e}^{i ∠ [ {\hat{s}}_{j} ( {τ}_{j} ) \cdot {\hat{s}}_{k} ( {τ}_{k} ) ]} \cdot \sqrt{\vert {\hat{s}}_{j} ( {τ}_{j} ) \cdot {\hat{s}}_{k} ( {τ}_{k} ) \vert} & ( 7 ) \\ = ( {\sum }_{j = 1}^{N} {e}^{i ∠ {\hat{s}}_{j} ( {τ}_{j} )} \sqrt{\vert {\hat{s}}_{j} ( {τ}_{j} ) \vert} ) ( {\sum }_{k = 1}^{N} {e}^{i ∠ {\hat{s}}_{k} ( {τ}_{k} )} \sqrt{\vert {\hat{s}}_{k} ( {τ}_{k} ) \vert} ) & ( 8 ) \\ = {( {\sum }_{j = 1}^{N} {e}^{i ∠ {\hat{s}}_{j} ( {τ}_{j} )} \sqrt{\vert {\hat{s}}_{j} ( {τ}_{j} ) \vert} )}^{2} & ( 9 )\end{matrix}$$

where y_{GDMAS} is the output of the GDMAS algorithm and ∠ŝⱼ represents the phase of the corresponding analytic signal. Equation (7) shows that, as in the DMAS algorithm, the square root of the absolute value of the multiplied amplitudes is summed. However, the sign function for the real signals of the DMAS algorithm is replaced by the complex exponential of the phase for the complex analytic signals of the GDMAS algorithm. The complex exponential can be thought of as a generalized sign function. Instead of only taking the values +1 or −1, the complex exponential may take any value on a unit circle in the complex plane.

Note that equation (9) is equivalent to equation (7), but there is only one summation. Removal of one summation greatly reduces the computation time of equation (9) relative to the DMAS algorithm, even though equation (9) considers the entire correlation matrix of the signals in reception.

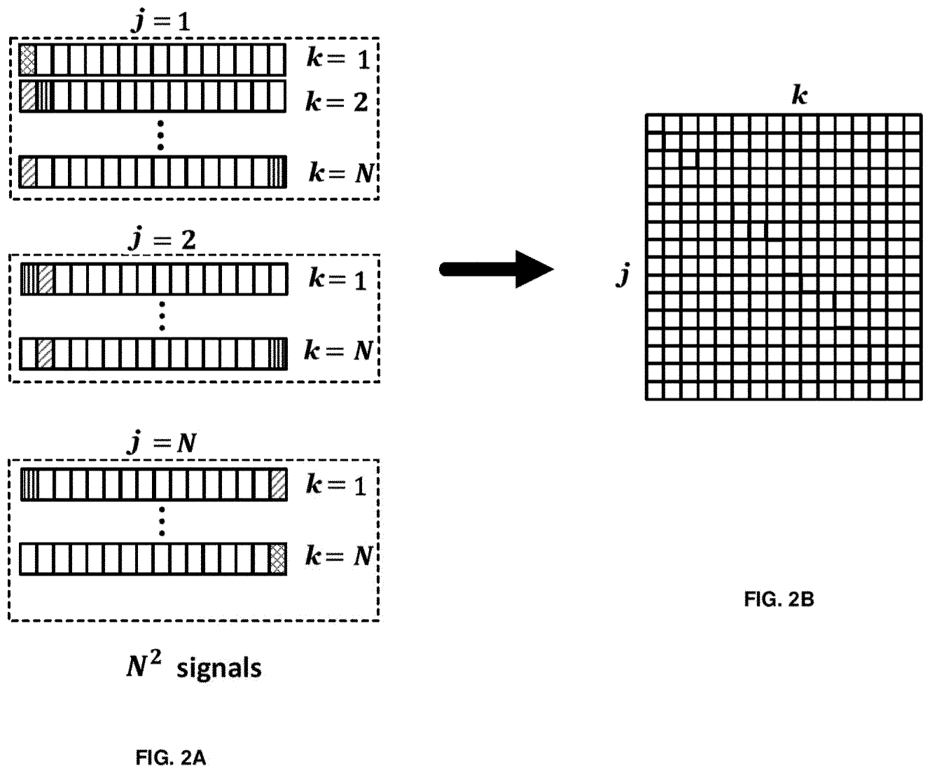

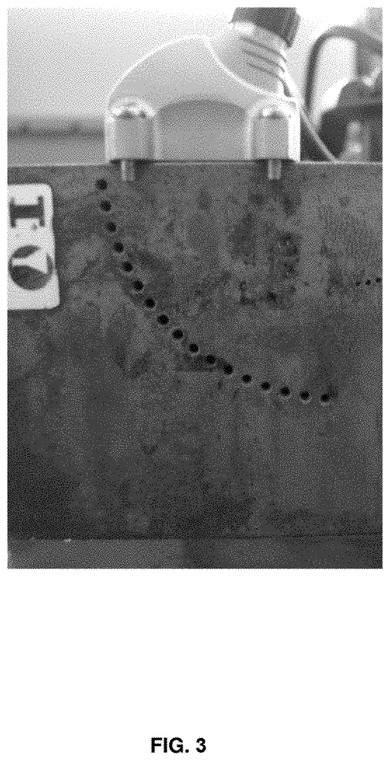

FIGS. 2A and 2B are pictorial representations of the summations used in the GDMAS algorithm. FIG. 2A illustrates that summations for j=1, 2 . . . N are all from k=1 to k=N. FIG. 2B illustrates a correlation matrix of the summations in jk space. Summation occurs for all the cells of the matrix (shown unshaded in FIG. 3B), including the diagonal elements (autocorrelations). Moreover, the summations for jk are repeated for kj. The total number of summed signals in GDMAS (N²) is therefore larger by more than a factor of 2 than the number of summed signals in DMAS (N(N−1)/2), and the autocorrelation terms are included, thereby removing undue emphasis on the cross-correlation terms. Yet, despite the larger number of summed signals, the computation time of GDMAS is less than that of DMAS.

When the imaging method is FMC TFM imaging, the summation operation is performed over all possible products of waveforms present in the FMC matrix:

$${y}_{GDMAS} = {( {\sum }_{m = 1}^{T} {\sum }_{n = 1}^{R} {e}^{i ∠ {\hat{s}}_{mn} ( {τ}_{mn} )} \sqrt{\vert {\hat{s}}_{mn} ( {τ}_{mn} ) \vert} )}^{2} \qquad ( 10 )$$

where ŝₘₙ is the signal recorded with delay τₘₙ by the n^{th} element when the m^{th} element is transmitting, T is the number of transmitting elements and R is the number of receiving elements. The product RT is equal to the number of waveforms N.

In step 3 of the GDMAS algorithm, the order of the method is raised to any real positive number Q. Instead of having a fixed order of 2 shown in equation (9), we now have:

$${y}_{GDMAS} = {( {\sum }_{j = 1}^{N} {e}^{i ∠ {\hat{s}}_{j} ( {τ}_{j} )} {\vert {\hat{s}}_{j} ( {τ}_{j} ) \vert}^{\frac{1}{Q}} )}^{Q} \qquad ( 11 )$$

Although equation (11) may be applied for any real number Q, for integer values of Q the equations are equivalent to summations providing correlation between Q signals. Equations (9) and (10) are equivalent to Q=2 and provide correlation between two signals A and B, where the summed multiplications are AA, AB, BA and BB. For Q=3, equation (11) provides correlation between three signals A, B and C, where the summed multiplications are AAA, AAB, AAC, ABA, ABB, ABC . . . etc. Equation (11)may provide similar correlations for Q=4, 5 . . . .

Note that the computational complexity of the exponential calculation is proportional to log(Q), and therefore rises slowly as Q is increased. It is also noted that the exponential computation time is small compared to the summation computation time.

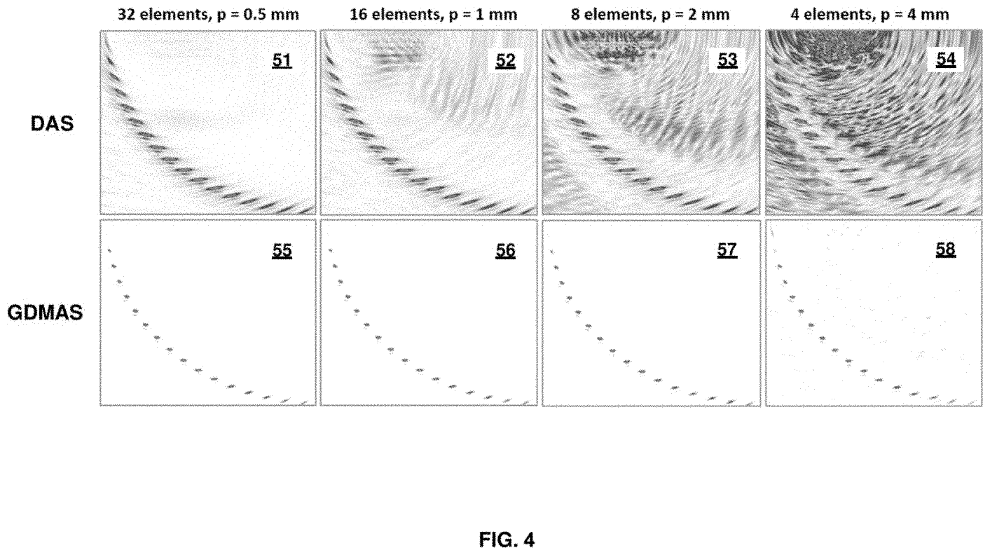

FIG. 3 is a photograph of an ultrasound array probe on a calibration block comprising a series of side-drilled holes having 2 mm diameter. FIG. 4 shows TFM images of the side-drilled holes created with the DAS algorithm (images 51, 52, 53 and 54) compared with images created with the GDMAS algorithm (images 55, 56, 57 and 58). Images 51 and 55 correspond to the same data acquired with a 32 element probe array having element spacing, p=0.5 mm. For images 52 and 56, data was acquired with only 16 of the 32 elements transmitting and receiving, corresponding to element spacing p=1 mm. For images 53 and 57, data was acquired with only 8 of the 32 elements transmitting and receiving, corresponding to element spacing p=2 mm. For images 54 and 58, data was acquired with only 4 of the 32 elements transmitting and receiving, corresponding to element spacing p=4 mm.

It can be seen that the images processed with GDMAS have clearly superior quality to those processed with DAS. Moreover, the marked deterioration in quality and resolution of the image for the DAS algorithm as the element spacing increased was not observed with the GDMAS algorithm. It should be noted that the computation time was similar in all cases for the GDMAS and DAS algorithms.

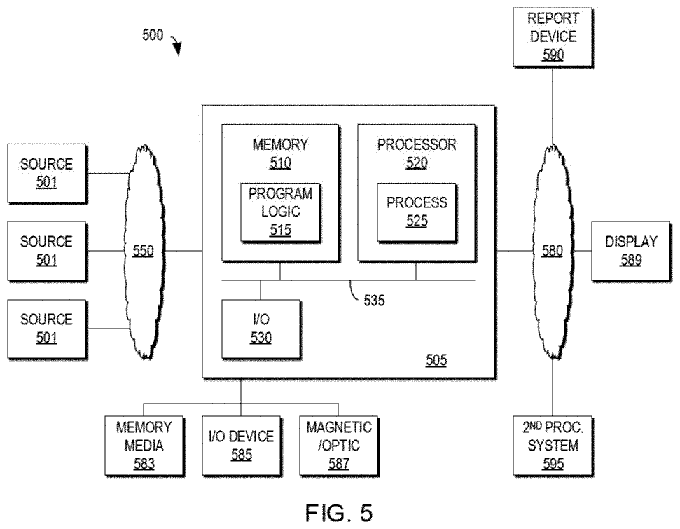

FIG. 5 is a block diagram of an example embodiment apparatus 505 for acquiring phased array ultrasonic testing data leveraging the principle of acoustic reciprocity according to an example embodiment of the present invention. The apparatus 505 may be part of a system 500 and includes memory 510 storing program logic 515, a processor 520 for executing a process 525, and a communications I/O interface 530, connected via a bus 535. The exemplary apparatus 505 is discussed only for illustrative purpose and should not be construed as a limitation on the embodiments or scope of the present disclosure. In some cases, some devices may be added to or removed from a computer system 500 based on specific situations.

Processing may be implemented in hardware, software, or a combination of the two. Processing may be implemented in computer programs executed on programmable computers/machines that each includes a processor, a storage medium or other article of manufacture that is readable by the processor (including volatile and non-volatile memory and/or storage elements), at least one input device, and one or more output devices. Program code may be applied to data entered using an input device to perform processing and to generate output information.

In some embodiments, the system may be embodied by one or more programmable processors executing one or more computer programs to perform the functions of the system. In some other embodiments, all or part of the system may be implemented as special purpose logic circuitry (e.g., a field-programmable gate array (FPGA) and/or an application-specific integrated circuit (ASIC)). In some other embodiments, all or part of the system may be implemented using electronic hardware circuitry that include electronic devices such as, for example, at least one of a processor, a memory, a programmable logic device or a logic gate.

In one embodiment, the methods described herein are not limited to the specific examples described. In a further embodiment, rather, any of the method steps may be re-ordered, combined or removed, or performed in parallel or in serial, as necessary, to achieve the results set forth above.

In some embodiments, the system may be implemented, at least in part, via a computer program product, (e.g., in a non-transitory machine-readable storage medium such as, for example, a non-transitory computer-readable medium), for execution by, or to control the operation of, data processing apparatus (e.g., a programmable processor, a computer, or multiple computers)). In certain embodiments, each such program may be implemented in a high level procedural or object-oriented programming language to communicate with a computer system. In certain other embodiments, however, the programs may be implemented in assembly or machine language. In some embodiments, the language may be a compiled or an interpreted language and it may be deployed in any form, including as a stand-alone program or as a module, component, subroutine, or other unit suitable for use in a computing environment. In some other embodiments, a computer program may be deployed to be executed on one computer or on multiple computers at one site or distributed across multiple sites and interconnected by a communication network.

The methods and apparatus of this invention may take the form, at least partially, of program code (i.e., instructions) embodied in tangible non-transitory media, such as floppy diskettes, CD-ROMs, hard drives, random access or read only-memory, or any other machine-readable storage medium. When the program code is loaded into and executed by a machine, such as the computer of FIG. 5, the machine becomes an apparatus for practicing the invention. When implemented on one or more general-purpose processors, the program code combines with such a processor to provide a unique apparatus that operates analogously to specific logic circuits. As such, a general purpose digital machine can be transformed into a special purpose digital machine. In some other embodiment, a non-transitory machine-readable medium may include but is not limited to a hard drive, compact disc, flash memory, non-volatile memory, volatile memory, magnetic diskette and so forth but does not include a transitory signal per se.

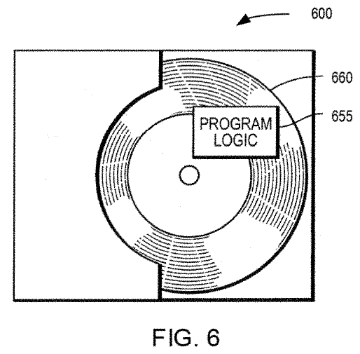

FIG. 6 is a block diagram of a computer program product 600 including program logic 655, encoded on a computer-readable medium 660 in computer-executable code configured for acquiring phased array ultrasonic testing data leveraging the principle of acoustic reciprocity according to an example embodiment of the present invention. The logic for carrying out the method may be embodied as part of the aforementioned system, which is useful for carrying out a method described with reference to embodiments shown. In one embodiment, program logic 655 may be loaded into memory and executed by processor. In a further embodiment, program logic 655 may also be the same program logic 655 on a computer readable medium.

Although the foregoing invention has been described in some detail for purposes of clarity of understanding, it will be apparent that certain changes and modifications may be practiced within the scope of the appended claims. The scope of the invention is limited only by the claims and the invention encompasses numerous alternatives, modifications, and equivalents. Numerous specific details are set forth in the above description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured. Accordingly, the above implementations are to be considered as illustrative and not restrictive, and the invention is not to be limited to the details given herein, but may be modified within the scope and equivalents of the appended claims.

Various exemplary embodiments of the present disclosure have been described with reference to the accompanying drawings. It may be appreciated that these example embodiments are provided only for enabling those skilled in the art to better understand and then further implement the present disclosure and not intended to limit the scope of the present disclosure in any manner. It should be noted that these drawings and description are only presented as exemplary embodiments and, based on this description, alternative embodiments may be conceived that may have a structure and method disclosed as herein, and such alternative embodiments may be used without departing from the principle of the disclosure as claimed in the present disclosure.

It may be noted that the flowcharts and block diagrams in the figures may illustrate the apparatus, method, as well as architecture, functions and operations executable by a computer program product according to various embodiments of the present disclosure. In this regard, each block in the flowcharts or block diagrams may represent a module, a program segment, or a part of code, which may contain one or more executable instructions for performing specified logic functions. It should be further noted that, in some alternative implementations, functions indicated in blocks may occur in an order differing from the order as illustrated in the figures. For example, two blocks shown consecutively may be performed in parallel substantially or in an inverse order sometimes, which depends on the functions involved. It should be further noted that each block and a combination of blocks in the block diagrams or flowcharts may be implemented by a dedicated, hardware-based system for performing specified functions or operations or by a combination of dedicated hardware and computer instructions.

The terms “comprise(s),” “include(s)”, their derivatives, and like expressions used herein should be understood to be open (i.e., “comprising/including, but not limited to”). The term “based on” means “at least in part based on”, the term “one embodiment” means “at least one embodiment”, and the term “another embodiment” indicates “at least one further embodiment”. Relevant definitions of other terms have been provided.

What is claimed is:

1. A method of forming an ultrasound image comprising:
receiving ultrasonic response signals;
deriving analytic signal values from the received ultrasonic response signals according to a Hilbert transform; and
performing a summation of a plurality of multiple signal value products each derived according to a respective set of analytic signal values.

2. The method of claim 1 wherein the corresponding set is a pair of the analytic signal values.

3. The method of claim 1 wherein the corresponding set is a group of Q analytic signal values, where Q is an integer number.

4. The method of claim 1 wherein the imaging method is a phased array ultrasound imaging method.

5. The method of claim 1 wherein the imaging method is a total focusing method.

6. The method of claim 1 wherein the summation further comprises summation of the signal product multiplied by a complex exponential of the phase of the signal product.

7. An ultrasound imaging system comprising:
an acquisition unit configured to receive ultrasonic response signals;
an imaging unit configured to derive analytic signal values from the received ultrasonic response signals according to a Hilbert transform and perform a summation of a plurality of multiple signal value products each derived according to a respective set of analytic signal values.

8. The system of claim 7 further comprising an ultrasonic probe configured to derive the signal value products according to a pair of analytic signal values.

9. The system of claim 7 wherein the imaging unit is further configured to derive the signal value products according to Q analytic signal values, where Q is an integer number

10. The system of claim 7 wherein the imaging unit is further configured to generate an image according to a total focusing method.

11. The system of claim 7 wherein the imaging unit is further configured to multiply the signal product by a complex exponential of the phase of the signal product

12. A computer program product having a non-transitory computer readable medium with computer program code stored thereon that, when executed on a processor, causes the process to forming an ultrasound image, the computer program code comprising:
computer program code for deriving analytic signal values from received ultrasonic response signals according to a Hilbert transform; and
computer program code for performing a summation of a plurality of multiple signal value products each derived according to a respective set of analytic signal values.
